(19) United States
(12) Patent Application Publication
(10) Pub. No.: US 2023/0277096 A1
(43) Pub. Date: Sep. 7, 2023

(54) MULTIMODAL, FULLY IMPLANTABLE, AND OPTICALLY FUNCTIONALIZED BIOSENSORS

(71) Applicant: The Texas A&M University System, College Station, TX (US)

(72) Inventors: Jesse M. Fine, College Station, TX (US); Gerard L. Coté, College Station, TX (US); Michael J. McShane, College Station, TX (US)

(21) Appl. No.: 18/115,587

(22) Filed: Feb. 28, 2023

Related U.S. Application Data
(60) Provisional application No. 63/314,908, filed on Feb. 28, 2022.

(51) Int. Cl. A61B 5/145; A61B 5/1486

(52) U.S. Cl. CPC A61B 5/14532; A61B 5/14865; A61B 5/1451

(57) ABSTRACT

In an embodiment, the present disclosure pertains to an analyte detection system. In some embodiments, the system includes a biosensor operable to be implanted into a media and a transmitter having a light source. In some embodiments, the transmitter is operable to be external to the media and operable to receive photoluminescence outputs back from the biosensor to determine properties of an analyte. In an additional embodiment, the present disclosure pertains to an analyte detection system. In some embodiments, the system includes a biosensor operable to be implanted into a media and a transmitter having a light source. In some embodiments, the transmitter is operable to be external to the media and operable to receive photoluminescence outputs back from the biosensor to determine properties of analytes. In a further embodiment, the present disclosure pertains to a biosensor having a plurality of discrete compartments in a barcode configuration.

### CROSS-REFERENCE TO RELATED APPLICATIONS

This patent application claims priority from, and incorporates by reference the entire disclosure of, U.S. Provisional Application 63/314,908 filed on Feb. 28, 2022.

### STATEMENT REGARDING FEDERALLY SPONSORED RESEARCH

This invention was made with government support under 1648451 awarded by the National Science Foundation. The government has certain rights in the invention.

### TECHNICAL FIELD

The present disclosure relates generally to biosensors and more particularly, but not by way of limitation, to multimodal, fully implantable, and optically functionalized biosensors.

### BACKGROUND

This section provides background information to facilitate a better understanding of the various aspects of the disclosure. It should be understood that the statements in this section of this document are to be read in this light, and not as admissions of prior art.

Multianalyte biosensors have been of interest in various applications, especially in healthcare areas that target a broad range of biomarkers related to various pathological diseases. However, successful examples of such sensors generally utilize electrochemical transducers, a fact which limits their use as implants due to the presence of interferents in vivo, the difficulty of transdermal signal transduction, and the difficulty of miniaturizing the electronics into an integrated biocompatible form factor suitable for implantation.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to an analyte detection system. In some embodiments, the system includes a biosensor operable to be implanted into a media and a transmitter having a light source. In some embodiments, the transmitter is operable to be external to the media and operable to receive photoluminescence outputs back from the biosensor to determine properties of an analyte. In some embodiments, the biosensor includes a plurality of discrete compartments in a barcode configuration. In some embodiments, each discrete assay includes at least one of an oxygen sensing assay and a glucose sensing assay. In some embodiments, at least one discrete compartment of the plurality of discrete compartments includes at least one glucose-responsive compartment. In some embodiments, at least one glucose-responsive compartment includes glucose-responsive microparticles for detection of glucose and oxygen. In some embodiments, at least one glucose-responsive compartment includes glucose-responsive microparticles having alginate microparticles for detection of glucose and oxygen. In some embodiments, the glucose-responsive microparticles include Pd(II) meso-tetra (sulfophenyl) tetrabenzoporphyrin sodium salt (PdBP). In some embodiments, glucose oxidase (GOx) and catalase (CAT) are included in fabrication of the glucose-responsive microparticles. In some embodiments, at least one discrete compartment of the plurality of discrete compartments includes at least one glucose-insensitive compartment. In some embodiments, at least one glucose-insensitive compartment includes glucose-insensitive particles for detection of oxygen. In some embodiments, at least one glucose-insensitive compartment includes glucose-insensitive particles having alginate microparticles for detection of oxygen. In some embodiments, the glucose-insensitive particles include Pd-meso-tetra(4-carboxyphenyl) porphyrin (PdP). While embodiments discussed above included at least one glucose-insensitive compartment, it should be noted that this is for illustrative purposes only. In some embodiments, the compartment can include, for instance, a glucose-sensitive compartment that responds in a different way than that of the glucose-responsive compartment. As such, the glucose-insensitive compartment may contain and assay that response differently than the glucose-responsive compartment, even if the assay is not specifically glucose-insensitive.

In an additional embodiment, the present disclosure pertains to an analyte detection system. In some embodiments, the system includes a biosensor operable to be implanted into a media and a transmitter having a light source. In some embodiments, the transmitter is operable to be external to the media and operable to receive photoluminescence outputs back from the biosensor to determine properties of analytes. In some embodiments, the biosensor includes a plurality of discrete compartments in a barcode configuration. In some embodiments, the biosensor includes a plurality of many assays, each in their own compartment. In some embodiments, the biosensor includes a plurality of many assays, each their own compartment, and additionally, can be repeated (e.g., the assays can appear in more than one compartment). In some embodiments, each discrete compartment of the plurality of discrete compartments includes at least one of a first analyte sensing assay or a second analyte sensing assay. In some embodiments, a first discrete compartment of the plurality of discrete compartments includes microparticles for detection of a first analyte and a second discrete compartment of the plurality of discrete compartments includes microparticles for detection of a second analyte. In some embodiments, a first discrete compartment of the plurality of discrete compartments includes microparticles for detection of a first analyte and a second analyte and a second discrete compartment of the plurality of discrete compartments includes microparticles for detection of the second analyte. In some embodiments, the biosensor has a barcode configuration having functionality through two modalities that relate interstitial analyte level to emitted photoluminescence. In some embodiments, the biosensor includes at least one sensing chemistry within a domain of the biosensor. In some embodiments, the biosensor has a stacked cylindrical design. In some embodiments, the media is skin.

In a further embodiment, the present disclosure pertains to a biosensor having a plurality of discrete compartments in a barcode configuration. In some embodiments, a first discrete compartment of the plurality of discrete compartments includes a glucose-responsive compartment. In some embodiments, a second discrete compartment of the plurality of discrete compartments includes a glucose-insensitive compartment. In some embodiments, the glucose-responsive compartment includes glucose-responsive microparticles for detection of glucose and oxygen. In some embodiments, the glucose-responsive compartment includes glucose-responsive microparticles having alginate microparticles for detection of glucose and oxygen. In some embodiments, the glucose-insensitive compartments include glucose-insensitive particles for detection of oxygen. In some embodiments, the glucose-insensitive compartments include glucose-insensitive particles having alginate microparticles for detection of oxygen.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIGS. 1A-1B illustrate a schematic diagram of a barcode hydrogel platform. FIG. 1A shows the 2×2 array barcode hydrogel sensor. FIG. 1B shows the composition and structure of the glucose and oxygen responsive microparticles.

FIGS. 2A-2E illustrate a fabrication process flow of a barcode hydrogel sensor. FIG. 2A shows loaded poly(ethylene glycol) diacrylate (PEGDA) precursor solution into the bottom mold. FIG. 2B shows the top mold was aligned with the bottom mold. The PEGDA solution was crosslinked by ultraviolet (UV) exposure. FIG. 2C shows removal of the top mold. A discrete 2×2 array of compartments was formed in the hydrogel. FIG. 2D shows the multi-sensing assays (A: glucose, B: oxygen) mixed with PEGDA solution were pipetted into each compartment. FIG. 2E shows the oxygen and glucose sensing assays were crosslinked by UV exposure. A and B represent glucose (Pd(II) meso-tetra (sulfophenyl) tetrabenzoporphyrin sodium salt; PdBP) and oxygen (Pd-meso-tetra(4-carboxyphenyl) porphyrin; PdP), respectively.

FIG. 3 illustrates time-lapse average photoluminescence intensity changes of the oxygen and glucose-responsive compartments. The barcode sensor was immersed in glucose solution with a concentration of 100 mg/dL.

FIGS. 4A-4B illustrate phosphorescence lifetime response of the barcode hydrogel sensor under different oxygen concentrations. FIG. 4A shows phosphorescence lifetime changes of the glucose (PdBP) and oxygen (PdP) responsive assays under different oxygen concentrations. FIG. 4B shows normalized phosphorescence lifetime ratio with the two types of sensing compartments. The circles represent the average phosphorescence lifetime response from the glucose-responsive compartments in three samples tested at the same time in the same flow cell, while the squares represent the glucose-insensitive compartments tested independently. Error bars represent the associated standard deviations.

FIG. 5 illustrates phosphorescence lifetime response of the barcode hydrogel sensor under different glucose concentrations. Circles represent an average of the phosphorescence lifetime response from the glucose-responsive compartments of three independent samples at the same time in the same flow cell, while squares represent the phosphorescence lifetime response of glucose-insensitive compartments from a single barcode sample. Error bars represent the standard deviations.

FIGS. 6A-6C illustrate stability assessment of the glucose response of the barcode hydrogel sensor. FIG. 6A shows each point represents an average of three samples. The phosphorescence lifetime was only collected from the glucose-insensitive compartment of each barcode sensor. Each data point represents an average of the phosphorescence lifetime response of three identical samples. Bar chart of the mean phosphorescence lifetime at 0 and 200 mg/dL glucose concentration over 10 cycles, respectively. FIG. 6B shows glucose responsive samples and FIG. 6C shows glucose-insensitive samples. Error bars represent 95% confidence intervals.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Multianalyte biosensors have been of interest in various applications, especially in healthcare areas that target a broad range of biomarkers related to various pathological diseases. However, successful examples of such sensors generally utilize electrochemical transducers, a fact which limits their use as implants due to the presence of interferents in vivo, the difficulty of transdermal signal transduction, and the difficulty of miniaturizing the electronics into an integrated biocompatible form factor suitable for implantation. The present disclosure demonstrates an attractive alternative for multiplexed sensing. Described herein is an optical sensing platform based on oxidoreductase enzymes coupled with optically distinct oxygen-sensitive metalloporphyrin phosphors which are immobilized within nanofilm-coated alginate microparticles; these are embedded in discrete compartments of a single miniature biocompatible hydrogel to achieve a “barcode” sensor device. Continuous multiplex oxygen and glucose monitoring were demonstrated through the measurement of phosphorescence lifetime changes of the metalloporphyrin phosphors when exposed to different environmental conditions. As expected, the phosphorescence lifetime exhibited a negative correlation with increases in oxygen concentration for all compartments. In contrast, the oxygen-responsive assays maintained a constant phosphorescence lifetime and the glucose-responsive assays showed increased phosphorescence lifetime when the glucose concentration increased from 0 to 400 mg/dL. This observation confirmed the desired behavior without any apparent crosstalk between the adjacent oxygen- and glucose-responsive compartments. Further, the sensors demonstrated high stability during repeated glucose challenges over 10 cycles. These findings suggest that the hydrogel barcode optical sensing platform has potential for use in various situations requiring multiplexed chemical analysis such as in continuous medical monitoring.

Biosensing technologies capable of simultaneously measuring multiple biomarkers associated with chronic diseases such as cancer, diabetes, cardiovascular disease, and chronic kidney disease are important to the future of personalized medicine and telemedicine, offering the potential to improve the way healthcare is monitored and delivered. Multianalyte biosensors can provide even more comprehensive patient health insights that would allow for more efficient and tailored therapies that can lead to reduced healthcare costs and improved patient outcomes. In addition to their role in personalized medicine, multianalyte biosensors may also improve the accuracy and reliability of physiological measurements by providing redundancy, even in biosensor systems intended to track a single disease.

Various types of multianalyte biosensors have been reported with applications in biotechnology, the nutrition industry, environmental analysis, and healthcare, among others. Multianalyte biosensors used in healthcare applications target a wide range of biomarkers associated with various pathological conditions, such as cancer, cardiovascular disease, and diabetes; from various biofluids including blood, interstitial fluid, saliva, and tear fluid. Numerous signal transduction and detection techniques have been explored in the development of multianalyte biosensing. Of the various modalities, enzymatic electrochemical biosensors are among the most common kinds of multianalyte biosensors. These systems operate by detecting a by-product of the reaction of an enzyme and an analyte. Due to the worldwide prevalence of diabetes mellitus, multianalyte biosensors capable of the simultaneous measurement of glucose as one of the key target analytes are among the most widely studied.

Multianalyte electrochemical biosensors often use enzymes as the selective bioreceptors, enabling the specific detection of target analytes of interest. One such ex vivo electrochemical multianalyte biosensor based on the immobilization of multiple enzymes in an electrode array was demonstrated. Similarly, a multianalyte biosensor capable of glucose and uric acid detection using dual channels for individuals suffering from diabetes and gout was developed. Another multi-enzyme sensor succeeded in detecting L-lactate and glucose using silicon chips with flow channels and achieved linear analyte responses in physiological ranges. Similarly, a lactate and glucose dual biosensor using an electropolymerization biofabrication technique was also developed. Additionally, a microfluidic chip with a solid-state sensor capable of the simultaneous detection of glucose, urea, and creatinine in human blood serum was also developed. This system used enzymes immobilized in alginate microbeads along with a magnetic powder to create a modular system that electrochemically measures the concentrations of target analytes. Multianalyte biosensors are also very well suited for pathologies such as cardiovascular disease due to the multitude of possible biomarkers. Examples include immunoassays capable of the simultaneous detection of cardiac troponin I (cTnI) and C-reactive protein (CRP) and a multianalyte immunoassay capable of detecting MMP-9 and IL-6 for ischemic stroke detection.

While many multianalyte electrochemical biosensors perform well in ex vivo applications, they are generally not well suited for implantation applications due to the presence of interferents in vivo, the difficulty of transdermal signal transduction, and the difficulty of miniaturizing the electronics into an integrated biocompatible form factor suitable for implantation. Many electrochemical biosensors are vulnerable to interferences because the electrodes used for analyte detection are often not specific. These systems often rely on the enzyme-induced oxidation of the metabolite and reduction of the electrode, which is vulnerable to the generation of nonspecific signals. One such ex vivo electrochemical multianalyte biosensor based on the immobilization of multiple enzymes in an electrode array was demonstrated.

Some semi-implanted (percutaneous) electrochemical sensing products are commercially available; however, these also suffer from drawbacks such as invasiveness, high expense, and the need for frequent sensor replacements. For example, a continuous glucose monitoring (CGM) device lasts only about 10 days before it must be replaced. These sensors are required to be inserted beneath the skin to measure glucose levels and cost about $150 per sensor on average. Furthermore, all the devices are prone to the dislodging of the cannula from motion due to the soft tissue environment. Additionally, various drugs can falsely alter the glucose reading for CGMs. These drawbacks offer a lot of room for improvement in reliably providing glucose data to patients.

Optical implantable multianalyte biosensors present possible solutions to these issues. A multianalyte optical biosensor capable of detecting changes in glucose, uric acid, urea, and creatinine by fluorescent intensity was reported. In this system, hydrolase and oxidase enzymes, along with horseradish peroxidase were immobilized using a sol-gel method. However, the analyte detection capability of this array biosensor comes with a few challenges. The system depends on two different principles based on the pH change and the reduction of the Am red reagent. The photoluminescence intensity readout could be affected by the possible fluctuation of the environment and defocusing issues. Further, the short-wavelength (violet) excitation light has only very shallow penetration into skin, which further limits its application in implantable continuous multianalyte monitoring under deep dermal layer.

Herein, a multianalyte optical biosensor platform aimed at leveraging the benefits of optical sensors and overcoming some of the issues of electrochemical systems is presented. The sensing components are based on oxidoreductase enzymes coupled with oxygen-sensitive metalloporphyrin phosphors immobilized together within nanofilm-coated alginate microparticles. These sensing elements are embedded in discrete compartments of a barcode hydrogel implant made of poly(ethylene glycol) diacrylate (PEGDA).

The biosensing assay indirectly detects changes in analytes through oxidase-induced local oxygen depletion to produce changes in phosphorescence lifetime via the oxygen-sensitive phosphors. Therefore, by changing the selection of oxidase enzymes used in the assay, the system can be used to target various analytes of interest and in the development of multianalyte biosensors. Particularly, since the immobilized phosphors are collisionally quenched in the presence of oxygen, the reduction in oxygen results in an increase in phosphorescence lifetime that is used to calculate the concentration of the analyte of interest.

A key advantage of measuring phosphorescence lifetime instead of luminescence intensity is the capability to conduct transcutaneous interrogation more effectively without significant optical interference from autophotoluminescence and background noise. This is especially valuable in the development of implantable biosensors where scattering and light absorption from the surrounding tissue is likely to be a factor.

The present disclosure demonstrates the feasibility of a multianalyte optical sensing platform to encapsulate discrete assays into an implantable and biocompatible hydrogel system fabricated using a soft lithography technique. To illustrate the capability of the system to target unique analytes with minimal crosstalk, oxygen and glucose were selected as model target analytes. The time-lapse fluorescent intensity of the oxygen and glucose-sensing compartments was studied and analyzed under different oxygen concentration conditions. To measure the system response to glucose and oxygen changes, the phosphorescence lifetime as a function of oxygen and glucose concentration was also studied. Overall, this multiplexed and implantable barcode hydrogel platform appears to have strong potential for monitoring, management, and treatment of various pathological conditions and can be a valuable tool in personalized medicine.

The multianalyte optical sensing platforms of the present disclosure made use of Monte Carlo simulations to select a design for a fully implantable repeatable unit photoluminescent multianalyte optical sensing platform. The model was constructed via known optical properties, and then, possible designs were created and tested. The photoluminescence output as a function of device location was determined, as well as photoluminescence “efficiency” of the various designs. After determining designs that yielded the best results, the multianalyte optical sensing platform was further optimized.

Reference will now be made to more specific embodiments of the present disclosure and data that provides support for such embodiments. However, it should be noted that the disclosure below is for illustrative purposes only and is not intended to limit the scope of the claimed subject matter in any way.

Materials. Poly(ethylene glycol) diacrylate (PEGDA, average Mw ˜3.4 kDa) was purchased from Alfa Aesar (Haverhill, Mass., USA). 2,2-dimethoxy-2-phenylacetophenone (C₆H₅COC(OCH₃)₂C₆H5, >99%), 1-vinyl-2-pyrrolidinone (C₆H₉NO, >99%) were obtained from Sigma-Aldrich (St. Louis, Mo., USA). The two phosphors, Pd(II) meso-Tetra (sulfophenyl) Tetrabenzoporphyrin Sodium Salt and Pd(II) meso-Tetra(4-carboxyphenyl)porphine) were obtained from Frontier Scientific (Logan, Utah, USA). Glucose oxidase from Aspergillus Niger was obtained from BBI solutions (Cardiff, UK) while catalase from bovine liver was obtained from Calzyme Laboratories, Inc. (San Luis Obispo, Calif., USA). Alginic acid sodium salt, poly(allylamine hydrochloride) (PAH, average Mw ˜17.5 kDa), poly(sodium-4-styrenesulfonate) (PAH, average Mw 70 kDa), calcium carbonate, TRIZMA Base (Tris[hydroxymethyl]aminomethane), and dimethyl sulfoxide (DMSO) were purchased from Sigma Aldrich (St. Louis, Mo., USA). The surfactant, Sorbitan trioleate (SPAN 85), was obtained from Tokyo Chemical Industry (Tokyo, Japan), while polyoxyethylene sorbitan trioleate (TWEEN 85) was obtained from Sigma Aldrich (St. Louis, Mo., USA). 2,2,4-Trimethylpentane (isooctane) was purchased from Tokyo Chemical Industry (Tokyo, Japan). Tris HCl (Tris-(hydroxymethyl) aminomethane hydrochloride) was purchased from VWR (Radnor, Pa., USA). Finally, calcium chloride (CaCl₂) and β-D-glucose were purchased from Macron Fine Chemicals (Center Valley, Pa., USA).

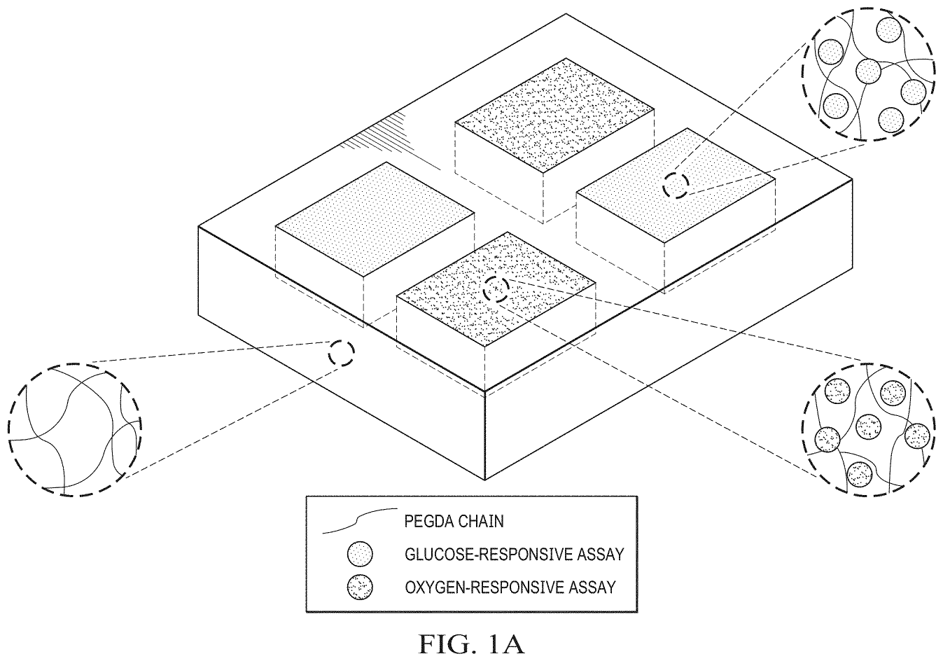

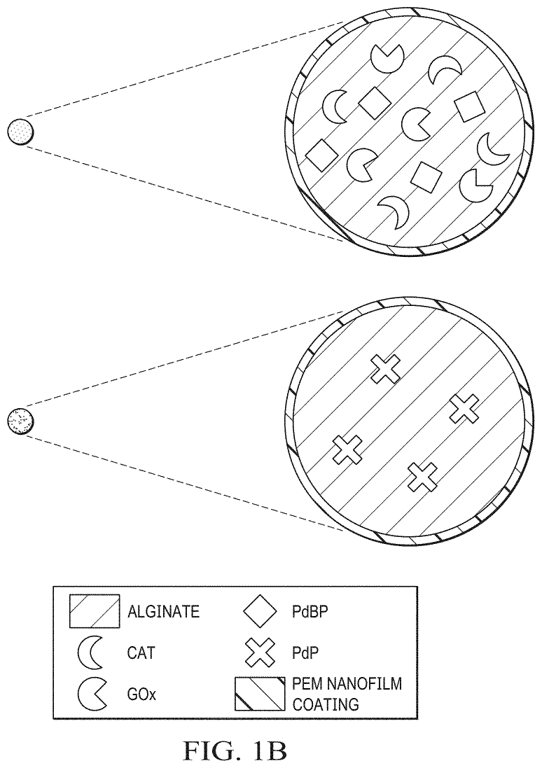

Oxygen and glucose sensing assays synthesis. FIG. 1A contains a schematic diagram of the barcode hydrogel sensor with discrete compartments containing oxygen and glucose sensing assays. Two groups of alginate microparticles were fabricated for use in the barcode sensors. One group of alginate microparticles was designed for the detection of both glucose and oxygen (glucose-responsive microparticles), while the second group of particles was designed only for the detection of oxygen (glucose-insensitive particles). Glucose oxidase (GOx) and catalase (CAT) were included in the fabrication of the glucose-responsive microparticles and excluded in the fabrication of glucose-insensitive particles (detailed below) to control glucose sensitivity. GOx serves as a highly selective glucose bioreceptor while CAT reduces hydrogen peroxide (H₂O₂), which is a reaction byproduct that is known to deactivate enzymes such as GOx.

All alginate microparticles were placed within compartments of the barcode construct, which contained four discrete compartments, consisting of two types of compartments, corresponding to the two types of particles (FIG. 1B). The compartments containing glucose-responsive microparticles will be further denoted as “glucose-responsive compartments”, while the compartments containing glucose-insensitive microparticles will be further denoted as “glucose-insensitive compartments”. For optical differentiation of both compartment types, two different porphyrin dyes, PdBP [Pd(II) meso-Tetra (sulfophenyl) Tetrabenzoporphyrin Sodium Salt] and PdP [Pd-meso-tetra(4-carboxyphenyl) porphyrin (PdTCPP/PdP), PdP] were included in each microparticle type, with PdP being included in glucose-insensitive particles and PdBP being included in the glucose-responsive microparticles.

An emulsion-based procedure was used to make both types of alginate microparticle droplets followed by crosslinking to form solid microparticles. Here, alginic acid was combined with the porphyrin molecules PdBP or PdP to create the aqueous phase of the emulsion. For glucose-responsive particles, both glucose oxidase (GOx) and catalase (CAT) were combined with this aqueous phase.

The alginate-dye-enzyme (or alginate-dye) mixture was then added to an organic phase of isooctane and emulsified using a homogenizer after the addition of surfactants TWEEN 85 and SPAN 85, which stabilize the emulsion by reducing the surface tension of the microparticle droplets as they crosslink within the alginate microparticles. After the crosslinking of the microparticles, a 5-bilayer alternating polyelectrolyte multilayer (PEM) thin films of poly (styrene sulfonate) (PSS) and poly (allylamine hydrochloride) (PAH) was deposited on the particles using layer-by-layer deposition to control glucose diffusion. The surface-modified microparticles were then suspended in 1 mL TRIS buffer solution (50 mM) and stored at 4° C. until use.

Barcode hydrogel sensor fabrication. The geometry of the multicompartment sensor and dimensions of the inner compartments were selected to optimize the ability of the optical readers to obtain consistent sensor readings with a strong signal-to-noise ratio. Narrower sensor geometries created difficulty in conducting measurements due to the spatial offset between the light emitting diode (LED) and photomultiplier tube (PMT) of the optical readers. However, the sensor is still small enough for a minimally invasive insertion procedure. As a reference, the only Food and Drug Administration (FDA)-approved fully implantable continuous glucose biosensor on the market is 3.5×18 mm which is still larger than the biosensor presented herein.

To create discrete compartments in a single barcode hydrogel, a bottom master mold and a top master mold were fabricated by replica molding polydimethylsiloxane (PDMS) from the master molds printed by a three-dimensional (3D) printer. The bottom and top master molds were designed such that the bottom PDMS master mold for the fabrication of the barcode hydrogel had arrays of hollow cuboid structures with the dimension of 3.5 mm in length, 3.1 mm in width, and 1 mm in height. The top PDMS master mold for the creation of the discrete compartments had arrays of bulge cuboids with dimensions of 1 mm in length, 0.8 mm in width, and 0.8 mm in height. The distance between each compartment was designed at 0.5 mm. Alignment markers were also designed and fabricated on the master molds to precisely match the bottom and top master molds. PDMS precursor was prepared by mixing the prepolymer and curing agent at a ratio of 10:1. The solution was well mixed and degassed before casting into the molds.

The hydrogel solution was prepared by mixing 10% (w/v) PEGDA and 2% (v/v) photo-initiator solution. The photo-initiator solution was prepared by adding 2,2-dimethoxy-2-phenylacetophenone into 1-vinyl-2-pyrrolidinone with a concentration of 100 mg/mL. The bottom and top PDMS master molds were pre-treated with oxygen plasma for 2 min to make the surface hydrophilic. Immediately after the surface treatment, the hydrogel solution was loaded into the bottom master mold. The top master mold was aligned on top of the bottom master mold with the help of the alignment markers. Then, the hydrogel polymerization was conducted by cross-linking under 365 nm ultraviolet (UV) exposure for 5 min. The barcode hydrogel with a 2×2 array of discrete compartments was peeled off the master molds and rinsed by deionized (DI) water (resistivity ˜18.2 MΩ/cm) for at least three times.

The oxygen and glucose sensing assays were mixed with the hydrogel solution at a ratio of 3:1. The well-dispersed hydrogel and the sensing assay mixtures were loaded into each compartment with a volume of 0.64 μL by a pipette followed by the polymerization of the mixed precursors under 365 nm ultraviolet (UV) for 5 min. The as-fabricated barcode hydrogel containing multiplexed sensing assays were then rinsed by DI water for at least three times and kept in DI water at 4° C.

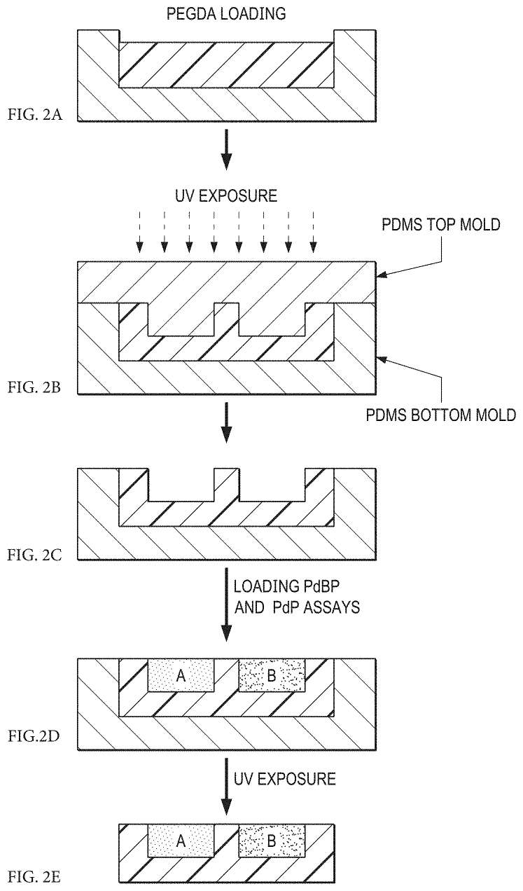

FIGS. 2A-2E illustrate a fabrication process flow of a barcode hydrogel sensor. FIG. 2A shows load PEGDA precursor solution into the bottom mold. FIG. 2B shows the top mold was aligned with the bottom mold. The PEGDA solution was crosslinked by UV exposure. FIG. 2C shows removal of the top mold. A discrete 2×2 array of compartments was formed in the hydrogel. FIG. 2D shows the multi-sensing assays (A: glucose, B: oxygen) mixed with PEGDA solution were pipetted into each compartment. FIG. 2E shows the oxygen and glucose sensing assays were crosslinked by UV exposure. A and B represent PdBP and PdP, respectively.

Testing system. Four barcode samples were tested in a single flow cell for each experiment. For each oxygen concentration, the barcode samples were tested for roughly 90 minutes to allow for the normalization of oxygen concentration and for the phosphorescence lifetime to reach a steady state. Absorbance and emission measurements were performed using an Infinite 200 PRO 96-well plate reader (Tecan, Männedorf, Switzerland). Radical crosslinking was initiated using a Blak-Ray B-100SP UV lamp from UVP (Upland, Calif., USA). Flow-through experiments were conducted using peristaltic pumps (L/S 7550 pump drive), pump heads (Easy Load 3), and precision tubing (L/S Norprene Tubing A60 G, L/S 13, 50 ft) purchased from MasterFlex (Gelsenkirchen, Germany). Oxygen concentrations were adjusted using mass flow controllers (Type 1179A General Purpose Mass-Flo Controller) and a pressure control unit (PR 4000 F) from MKS Instruments (Andover, Mass., USA). Optical interrogation of hydrogel samples was conducted using custom optical readers, described in previous studies.

Each reader contained a red or green LED (Lumileds LUXEON Rebel, λₑx=630 nm λₑx=530 nm for red and green wavelengths, respectively) for excitation and silicon photomultiplier tubes (SiPMT, SensL) for emission detection. During oxygen and glucose response testing, samples were housed in a custom-designed Delrin flow cell capable of holding four hydrogel samples and four optical readers. Changes in oxygen concentration during the oxygen response tests were verified using an OX-500 oxygen microsensor and PA2000 picoammeter (Unisense, Aarhus, Denmark).

Optical and fluorescent images. The optical and fluorescent images of the barcode hydrogel sensor were acquired using a Leica SP8 confocal microscope. The time-lapse fluorescent images of the barcode hydrogel sensor with oxygen and glucose sensing compartments were analyzed. A chamber was created by two cover glasses with an adhesive spacer to confine the specimen. The barcode hydrogel sensor sample was put inside the chamber. DI water was first injected into the chamber so that the sample was immersed in an ambient oxygen concentration environment as a control. Fresh glucose solution with a concentration of 100 mg/dL was then added to the chamber. The glucose sensing assays were excited by 630 nm and the wavelength of the emission filter ranges from 750 nm to 800 nm. The oxygen sensing assays were excited by 530 nm and the wavelength of the emission filter ranges from 660 nm to 700 nm. The total duration for taking the time-lapse fluorescent images was set as 2 h with a 654 s interval. The mean fluorescent intensity of the glucose and oxygen sensing compartment with the same area of the region of interest was analyzed by Leica Application Suite X core 3.7.4.23463.

Microparticle concentration and size distribution analysis. The size distribution of the microparticles was characterized by a Cellometer (Nexcelom Bioscience, Massachusetts, USA). The sample solution was prepared by diluting the microparticle stock solution at a ratio of 1:10 by TRIS buffer solution (50 mM) and resuspended in solution before imaging. The concentration as a function of the size of the microparticles was then plotted and analyzed.

Statistical analysis. All data are processed and presented in the form of mean±standard deviation. Student's t-test and one-way analysis of variance test were used to compare datasets. A p-value of less than 0.05 was considered statistically significant at a 95% confidence level.

Bright field and photoluminescence images. The dimensions of the single barcode hydrogel were confirmed by a top-view grayscale image and complementary color images were analyzed. The glucose-responsive compartments appear dark green, while the oxygen-responsive compartments appear red in a 2×2 array. There is less photoluminescence intensity in the center since the microparticles attach to the sidewall of the compartment due to surface tension.

The time-lapse photoluminescence images in the oxygen and glucose-responsive compartments were taken when the barcode hydrogel sensor was immersed in 100 mg/dL glucose solution in a sealed chamber. The photoluminescence intensity of the compartment was calculated by the average photoluminescence intensity in the compartment region of interest minus the average photoluminescence intensity in the dark background.

It is worth noting that the natural lifetime range of PdP is much greater than that of PdBP. The natural lifetime range of PdP in ˜0% oxygen concentrations is roughly 586 μs while PdBP is 203 μs. Therefore, the responses are reported as a percentage change rather than an absolute phosphorescence lifetime change.

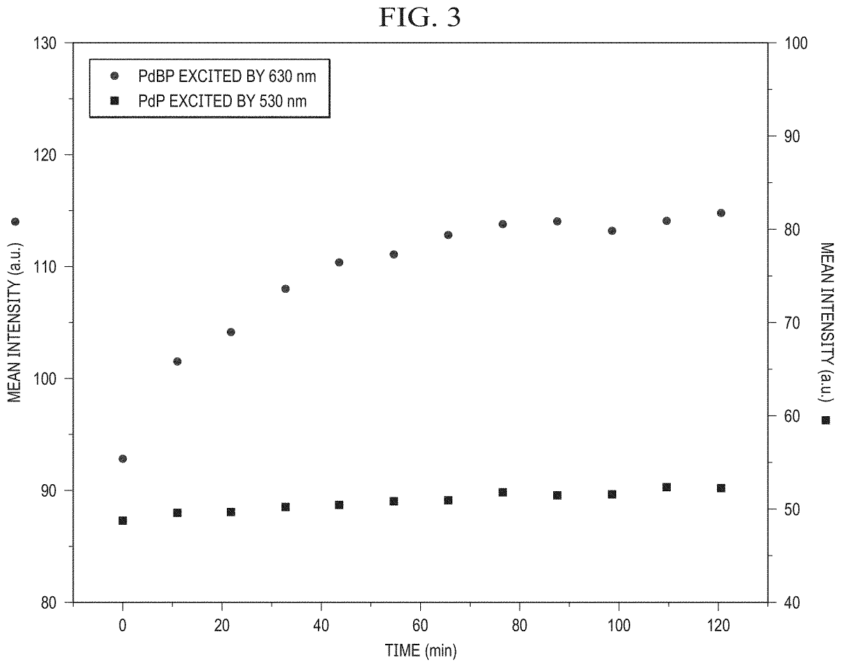

As shown in FIG. 3, the mean fluorescent intensity of the glucose-responsive compartment increased by about 23.6% after exposure to glucose solution for 2 h. This change corresponds to a 7.8% decrease in oxygen concentration. The mean intensity started to saturate after around 90 min. This is likely due to the consumption of oxygen in the glucose-responsive compartment when glucose was added to the sealed environment, as this formed a low oxygen condition in the hydrogel matrix. When the porphyrin was quenched by oxygen, the fluorescent intensity increased as the oxygen concentration decreased.

When the local oxygen concentration became very low in the glucose-responsive compartment, the mean intensity leveled off as the oxygen concentration became constant. However, the mean intensity of the oxygen-responsive compartment showed only about a 7.2% increase, which corresponds to a 0.7% decrease in oxygen concentration. This suggests the oxygen concentration in the oxygen-responsive compartment was barely affected by the oxygen consumption in the nearby glucose-responsive compartments.

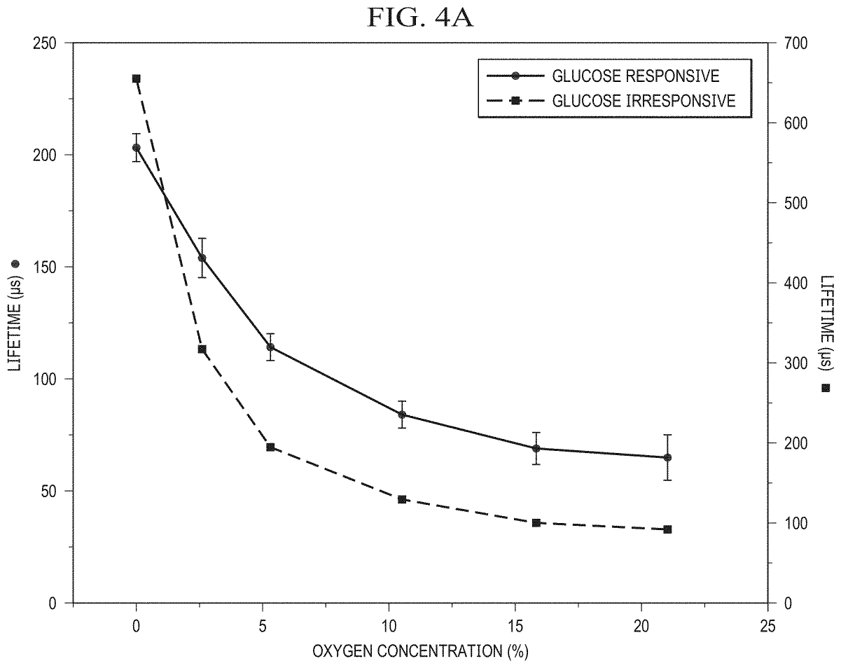

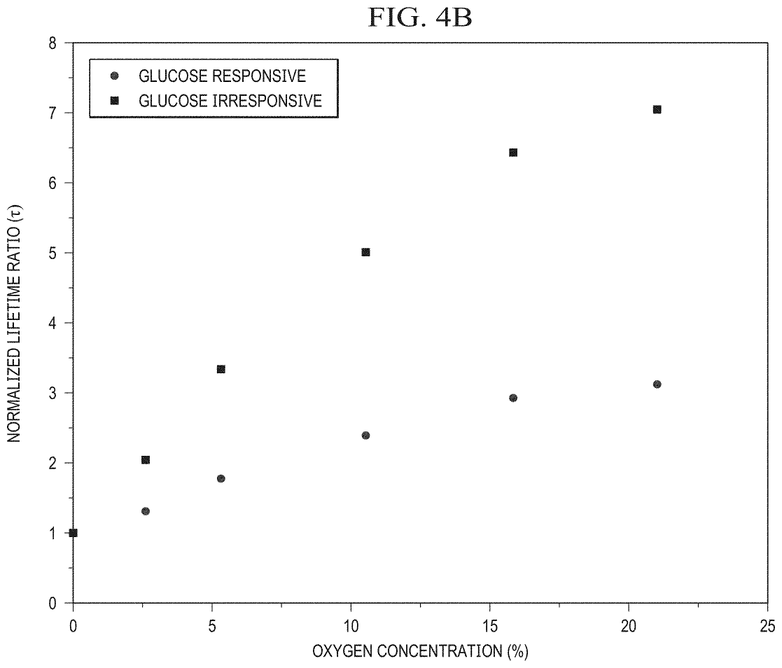

Oxygen response. The response of the barcode hydrogel sensors to the changes in oxygen concentrations was measured. Specifically, the phosphorescence lifetimes changes associated with both glucose-responsive and glucose-insensitive compartments were analyzed as a function of oxygen concentration. An oxygen concentration ranging from 0 to 21% (0-206.8 μM) was used, to represent oxygen concentrations up to ambient oxygen levels. The normalized phosphorescence lifetime ratio τ₀/τ is defined as the fraction of the phosphorescence lifetime τ₀ to at 0% oxygen concentration to the phosphorescence lifetime τ at a given oxygen concentration. The sensitivity of each barcode compartment to oxygen is quantified by the Stern-Volmer constant, Kₛᵥ, which was calculated as the slope of the normalized oxygen response graph in FIG. 4B.

Both PdBP and PdP compartments exhibited pronounced responses to the changes in oxygen concentration (FIG. 4A), as expected. The phosphorescence lifetime differed significantly between the two compartments due to the difference in the natural lifetime range of the phosphors contained in each compartment group. The barcode compartments that contained the PdBP (phosphor) and GOx showed a phosphorescence lifetime range of 65-203 μs (138 μs) and a Kₛᵥ value of 11.3 (% O₂)⁻¹*10⁻². Conversely, the oxygen sensitivity of PdP, which contained PdP (phosphor) and no GOx or CAT, had a much higher phosphorescence lifetime range of 93-655 μs (562 μs) and a Kₛᵥ value of 32.4 (% O₂)⁻¹. However, in both compartment types, oxygen sensitivity is greater in low-oxygen conditions and lower in greater-oxygen environments. A similar trend has been observed in other oxygen-sensing porphyrin systems.

It should be noted that although the phosphorescence lifetime range for PdP was greater than PdBP, the latter phosphor was selected for glucose sensing due to its red excitation wavelength (˜630 nm), which is within the “optical window” where light absorption by both proteins and water is low. Conversely, PdP has an excitation wavelength close to 530 nm, which would make in vivo excitation of the phosphor more difficult due to the absorption and scattering of light by pigments present in vivo. This phosphor was used in this study because it was commercially available and allows for an easy demonstration of the ability to encapsulate unique optical assays of very distinct excitation wavelengths. Obtaining phosphors that have distinct excitation wavelengths while remaining within the optical window can be challenging and may require custom synthesis of phosphors. However, for clinical translation of an optical implantable biosensor system that communicated with an external reader, it may be necessary to select phosphors that operate in the “optical window” (600-950 nm).

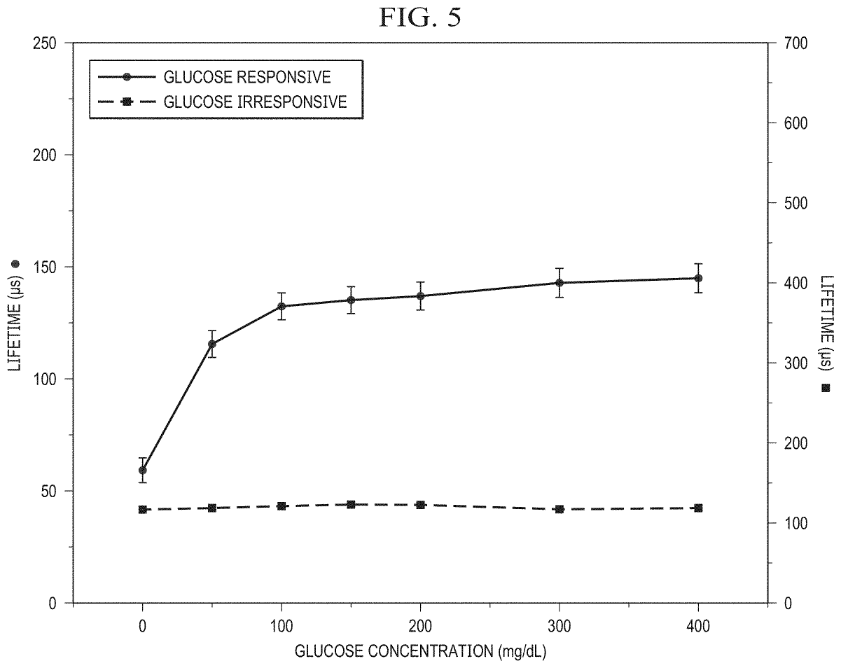

Glucose response under a fixed oxygen concentration. The glucose response was obtained by measuring the phosphorescence lifetime as a function of glucose concentration from the barcode sensors (FIG. 5). As expected, the PdBP compartments showed a much more pronounced response with a phosphorescence lifetime increase of 86 μs (59 μs to 145 μs) to changes in glucose concentration than PdP compartments, which only a phosphorescence lifetime increase of 2 μs (117 μs to 118 μs). These phosphorescence lifetime changes correspond with an 18.6% decrease in oxygen concentration (from 20.4% O₂ to 1.81% O₂) for the glucose-responsive compartments and a 0.2% decrease in oxygen concentration (from 18.3% O₂ to 18.1% O₂) for glucose-responsive compartments.

It is again worth noting that the natural lifetime range of PdP is much greater than that of PdBP as shown in FIGS. 4A-4B. As such, the lifetime responses during glucose challenges are better reported as a percentage change than an absolute phosphorescence lifetime change instead of lifetime changes. The glucose-sensitive compartments (with PdBP-containing microparticles) showed a 62% change in phosphorescence lifetime while the glucose-insensitive compartments (with PdP-containing microparticles) showed only a 0.3% change in phosphorescence lifetime. These glucose responses are as expected and are consistent with the time-lapse fluorescent intensity testing results mentioned in FIG. 3.

The presence of a very low response in Compartment PdP also demonstrates a low level of crosstalk in the barcode system since the glucose-sensitive compartment responded to the change in oxygen while the glucose-insensitive compartment did not show a significant response.

Although a significant increase in phosphorescence lifetime was observed from the PdBP compartments during glucose challenges, it does not achieve the maximum lifetime observed in the oxygen response tests despite increases in glucose concentration. This observed plateau in the lifetime response that is lower than the maximum achievable lifetime implies that the glucose response is enzyme-limited. In other words, the enzyme-induced oxygen depletion by all available enzymes does not consume all local oxygen within the barcode compartments. To achieve a higher lifetime range and achieve a higher lifetime range in future studies, the amount of GOx can be increased.

Stability assessments. Stability tests were conducted on the barcode sensor to evaluate the glucose response over multiple cycles. The barcode biosensors were exposed to 10 consecutive cycles of 0 mg/dL glucose and 200 mg/dL glucose. Phosphorescence lifetime measurements were made throughout this process and the percent change in the phosphorescence lifetime response over the cycles was calculated. The samples were exposed to each glucose concentration for 1 hour to allow enough time for diffusion-reaction to reach a dynamic equilibrium (˜10 mins) and data collection (30 mins). In total, the biosensor measurements for this experiment lasted for roughly 20 hours. Such a long-duration experiment is of particular importance in the design of implantable biosensors where the measurement may be performed for months after implantation. Hence, the long-term stability of the device is of high priority.

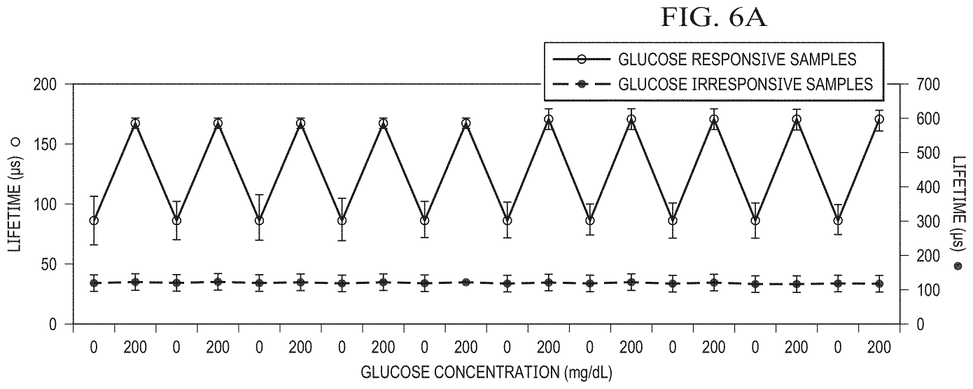

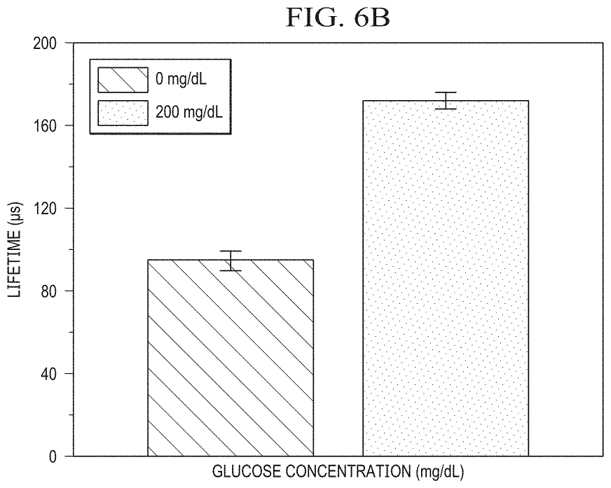

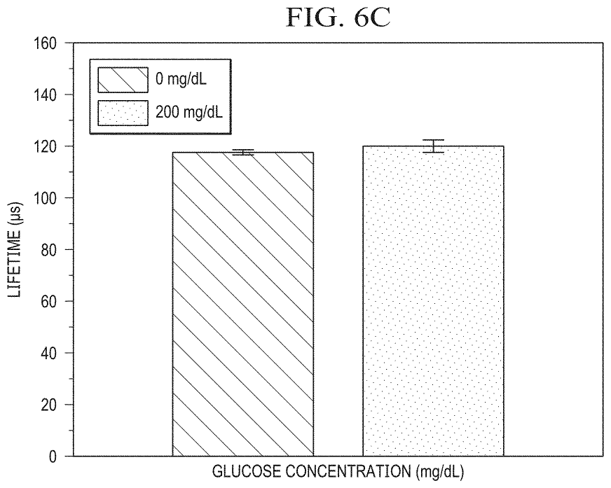

FIG. 6A and FIG. 6B show no significant changes in the phosphorescence lifetime of glucose sensors over the 10 cycles. Specifically, the phosphorescence responses to a 200 mg/dL glucose solution showed an average of 171.9 μs with a standard deviation of 3.7 μs in lifetime over 10 cycles while the phosphorescence responses to a 0 mg/dL glucose solution showed an average of 94.6 μs with a standard deviation of 3.7 μs in phosphorescence responses over 10 cycles. The relative stability of the barcode system may be due to the inclusion of catalase (CAT) in the sensing compartments. This enzyme has been reported to reduce the deactivation of GOx by H₂O₂, which is produced during glucose catalysis. It is also noteworthy that the glucose-insensitive compartments do not change lifetime when glucose levels change and they also exhibit a stable, steady emission lifetime (171.9±3.7 μs) over the duration of the experiment (FIG. 6C).

Conclusion. In this study, an implantable optical sensing platform was developed based on oxidoreductase enzymes coupled with oxygen-sensitive metalloporphyrin phosphors immobilized within nanofilm-coated alginate microparticles embedded in discrete compartments of a single barcode hydrogel. The barcode hydrogel optical sensing platform was fabricated by replica molding from the master molds printed by a 3D printer. Optical and photoluminescence images of the sensors were used to study and analyze the barcodes under different oxygen concentration conditions. Continuous oxygen and glucose monitoring were demonstrated by phosphorescence lifetime change of the metalloporphyrin phosphors in oxygen and glucose-responsive compartments, respectively. The oxygen-responsive assays showed a constant phosphorescence lifetime while the glucose-responsive assays showed increased phosphorescence lifetime with the glucose concentration increased from 0 to 400 mg/dL, which confirms there is no significant crosstalk between the nearby oxygen and glucose-responsive compartments. Finally, the sensors were found to have excellent stability of response when tested over 10 consecutive cycles of alternating 0 mg/dL and 200 mg/dL glucose. These results illustrate the effectiveness of these miniature hydrogel devices with compartments containing optical assays to serve as platforms for multianalyte sensing.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

What is claimed is:

1. An analyte detection system, the system comprising:
a biosensor operable to be implanted into a media; and
a transmitter comprising a light source, wherein the transmitter is operable to be external to the media and operable to receive photoluminescence outputs back from the biosensor to determine properties of an analyte.

2. The system of claim 1, wherein the biosensor comprises a plurality of discrete compartments in a barcode configuration.

3. The system of claim 2, wherein each discrete compartment comprises at least one of an oxygen sensing assay and a glucose sensing assay.

4. The system of claim 2, wherein at least one discrete compartment of the plurality of discrete compartments comprises at least one glucose-responsive compartment.

5. The system of claim 4, wherein the at least one glucose-responsive compartment comprises glucose-responsive microparticles comprising alginate microparticles for detection of glucose and oxygen.

6. The system of claim 5, wherein the glucose-responsive microparticles comprise Pd(II) meso-tetra (sulfophenyl) tetrabenzoporphyrin sodium salt (PdBP).

7. The system of claim 5, wherein glucose oxidase (GOx) and catalase (CAT) are included in fabrication of the glucose-responsive microparticles.

8. The system of claim 2, wherein the at least one discrete compartment of the plurality of discrete compartments comprises at least one glucose-insensitive compartment.

9. The system of claim 8, wherein the at least one glucose-insensitive compartments comprises glucose-insensitive particles comprising alginate microparticles for detection of oxygen.

10. The system of claim 9, wherein the glucose-insensitive particles comprise Pd-meso-tetra(4-carboxyphenyl) porphyrin (PdP).

11. An analyte detection system, the system comprising:
a biosensor operable to be implanted into a media; and
a transmitter comprising a light source, wherein the transmitter is operable to be external to the media and operable to receive photoluminescence outputs back from the biosensor to determine properties of analytes.

12. The system of claim 11, wherein the biosensor comprises a plurality of discrete compartments in a barcode configuration.

13. The system of claim 12, wherein each discrete compartment of the plurality of discrete compartments comprises at least one of a first analyte sensing assay or a second analyte sensing assay.

14. The system of claim 12, wherein a first discrete compartment of the plurality of discrete compartments comprises microparticles for detection of a first analyte, and wherein a second discrete compartment of the plurality of discrete compartments comprises microparticles for detection of a second analyte.

15. The system of claim 12, wherein a first discrete compartment of the plurality of discrete compartments comprises microparticles for detection of a first analyte and a second analyte, and wherein a second discrete compartment of the plurality of discrete compartments comprises microparticles for detection of the second analyte.

16. The system of claim 11, wherein the biosensor has a barcode configuration comprising functionality through two modalities that relate interstitial analyte level to emitted photoluminescence.

17. The system of claim 11, wherein the biosensor comprises at least one sensing chemistry within a domain of the biosensor.

18. The system of claim 11, wherein the biosensor comprises a stacked cylindrical design.

19. The system of claim 11, wherein the media is skin.

20. A biosensor comprising:
a plurality of discrete compartments in a barcode configuration;
wherein a first discrete compartment of the plurality of discrete compartments comprises a glucose-responsive compartment;
wherein a second discrete compartment of the plurality of discrete compartments comprises a glucose-insensitive compartment;
the glucose-responsive compartment comprising glucose-responsive microparticles comprising alginate microparticles for detection of glucose and oxygen; and
the glucose-insensitive compartments comprising glucose-insensitive particles comprising alginate microparticles for detection of oxygen.
